(19) United States
(12) Patent Application Publication
(10) Pub. No.: US 2025/0236518 A1
(43) Pub. Date: Jul. 24, 2025

(54) METHOD FOR PRODUCING HYDROGEN USING AT LEAST TWO BIOMETHANES

(71) Applicant: Iogen Corporation, Ottawa (CA)

(72) Inventor: Patrick J. Foody, Ottawa (CA)

(21) Appl. No.: 18/853,677

(22) PCT Filed: Apr. 7, 2023
(86) PCT No.: PCT/CA2023/050485

§ 371 Date: Oct. 2, 2024

Related U.S. Application Data
(60) Provisional application No. 63/362,786, filed on Apr. 11, 2022.

(51) Int. Cl. C01B 3/34; C01B 3/50

(52) U.S. Cl. CPC C01B 3/34; C01B 3/50; C01B 2203/0216; C01B 2203/0827; C01B 2203/1241

(57) ABSTRACT

A method of producing hydrogen that uses at least two batches of biomethane, where at least two of the biomethanes are from different sources, are produced from different feedstocks, are produced from different processes, and/or have different carbon intensities. The at least two batches of biomethane are distributed such that at least a portion of the first batch distributed to the feedstock, at least a portion of the second batch distributed to the fuel, and such that at least one of the biomethanes is distributed disproportionally between feedstock and fuel and/or such that biomethane in the feedstock has a different fractional make-up than biomethane in the fuel.

### TECHNICAL FIELD

The present disclosure relates to a method for producing hydrogen using at least two different biomethanes.

### BACKGROUND

Hydrogen (H₂) is a versatile energy carrier with exceptional energy density. It can be used as a fuel, as an industrial feedstock (e.g., to produce fuel, fuel intermediates, or chemical products), or in fuel cells (e.g., to generate heat and/or electricity). For example, hydrogen is commonly used in oil refining, ammonia production, methanol production, and steel production.

Today, most hydrogen is produced from the processing of fossil fuels, and thus is associated with high greenhouse gas (GHG) emissions. For example, hydrogen production based on the steam methane reforming (SMR) of natural gas can generate carbon dioxide both from the methane reforming reactions that generate hydrogen and from the combustion of natural gas, the latter of which is conducted to provide heat for hydrogen production (e.g., for the endothermic SMR). When this carbon dioxide is vented to the atmosphere, the hydrogen produced is often referred to as grey hydrogen. Grey hydrogen can have a carbon intensity (CI) that is greater than 80-90 gCO₂eq/MJ.

There is increasing interest in producing hydrogen having a relatively low carbon intensity. In one approach, hydrogen is produced from the electrolysis of water using renewable sources (e.g., wind, solar, geothermal, and/or hydroelectric power). The resulting hydrogen, which is often termed green hydrogen, can have a carbon intensity that is close to 0 gCO₂eq/MJ. Unfortunately, the production of green hydrogen is expensive relative to the production of grey hydrogen. In another approach, hydrogen production based on the methane reforming of natural gas is combined with carbon capture and storage (CCS), such that at least a portion of the carbon dioxide generated is captured and stored so as to prevent or delay its release to the atmosphere. The resulting hydrogen is often referred to as blue hydrogen. The carbon intensity of blue hydrogen can depend on whether the CCS includes only capturing the carbon dioxide produced from the methane reforming reactions, or also includes capturing the carbon dioxide produced from the combustion of natural gas (e.g., captured from the flue gas). In another yet another approach, hydrogen production based on methane reforming is combined with the use of biomethane. In general, biomethane can be used as feedstock for the hydrogen production and/or can be combusted to provide heat for the hydrogen production.

Using biomethane as fuel to provide heat for the hydrogen production can reduce the carbon intensity of the hydrogen produced (i.e., relative to using natural gas). Using biomethane as feedstock for the hydrogen production can reduce the carbon intensity of the hydrogen produced and/or can impart renewable content into the hydrogen produced. The renewable content can be provided as a renewable fuel and/or used to produce at least partially renewable fuel, fuel intermediates, or chemical product. For example, renewable hydrogen can be used to produce renewable ammonia, which can be used for energy storage, marine transportation, or sustainable agriculture. Alternatively, renewable hydrogen can be used to hydrogenate crude oil derived hydrocarbon in a fuel production process to make renewable or partially renewable fuel (e.g., see U.S. Pat. Nos. 8,658,026, 8,753,854, 8,945,373, 9,040,271, 10,093,540, 10,421,663, 10,723,621, 10,981,784). However, even when hydrogen is produced using biomethane, it may not necessarily be treated as clean and/or low carbon hydrogen under prevailing regulations and/or may not necessarily qualify for one or more carbon intensity based incentives (e.g., producer credits).

### SUMMARY

The present disclosure relates generally to a method for producing hydrogen using at least two biomethanes (e.g., biomethanes that are from different sources, are produced from different feedstocks, are produced from different processes, and/or have different carbon intensities), wherein at least a portion of one the biomethanes is associated with feedstock for methane reforming and at least a portion of another of the biomethanes is associated with fuel for the methane reforming.

In providing multiple biomethanes, the biomethanes can be distributed such that one or more of the biomethanes is distributed disproportionally between the feedstock for methane reforming and the fuel for methane reforming. For example, at least one of the biomethanes can be distributed disproportionally so as to provide the feedstock with biomethane having a certain fractional make-up and the fuel with biomethane having a different fractional make-up. Accordingly, the biomethane that is feedstock for the methane reforming and the biomethane that is fuel for the reforming can have different sustainability characteristics, weighted average carbon intensities, feedstocks, etc. In distributing one or more of the biomethanes disproportionally, the carbon intensity, types of feedstocks used, sustainability characteristics, and/or supply of each of the biomethanes can be used to maximize the amount of hydrogen (i.e., MJ) that meets a certain carbon intensity threshold (i.e., has a carbon intensity that is equal to or lower than the threshold), qualifies as clean or low carbon hydrogen, and/or qualifies for various incentives associated with a relatively low carbon intensity (i.e., under applicable regulations), for a given amount of biomethane provided (i.e., in MJ).

The present disclosure also relates to methods of producing fuel, chemical product, fuel or chemical intermediates, or any combination thereof using the hydrogen produced. For example, the methods can include methods of producing hydrogen, or methods of producing fuel (e.g., one or more fuels such as hydrogen, gasoline, diesel, jet fuel, methanol, ethanol, etc.), chemical product (e.g., methanol, ammonia, fertilizer, etc.), or intermediates (e.g., methanol, hydrogen, ammonia, ethanol, etc.).

In accordance with one aspect of the instant invention there is provided a method of producing hydrogen, the method comprising: (a) providing at least two batches of biomethane for producing hydrogen from a hydrogen production process, the at least two batches of biomethanes including a first batch of a first biomethane and a second batch of a second biomethane, the first and second biomethanes being produced from different feedstocks, being produced from different processes, having different carbon intensities, or any combination thereof, the hydrogen production process comprising: (i) subjecting feedstock to methane reforming to produce syngas, (ii) combusting fuel for producing heat for the methane reforming, and (iii) subjecting the syngas or a gas derived from the syngas to a hydrogen purification process; and (b) generating a quantity of hydrogen from the hydrogen production process using the at least two batches of biomethane, at least a portion of the first batch distributed to the feedstock, at least a portion of the second batch distributed to the fuel, a distribution of the at least two batches of biomethane between the feedstock and fuel providing the feedstock with biomethane having a first fractional make-up and the fuel with biomethane having a second fractional make-up, the first fractional make-up being different from the second fractional make-up.

In accordance with one aspect of the instant invention there is provided a method of producing hydrogen, the method comprising: (a) providing at least two batches of biomethane for producing hydrogen from a hydrogen production process, the at least two batches of biomethanes including a first batch of a first biomethane having a first carbon intensity and a second batch of a second biomethane having a second carbon intensity, the first and second biomethanes being different, the hydrogen production process comprising: (i) subjecting feedstock to steam methane reforming to produce syngas, (ii) combusting fuel for producing heat for the steam methane reforming, and (iii) subjecting the syngas or a gas derived from the syngas to a hydrogen purification process; and (b) generating a quantity of hydrogen from the hydrogen production process using the at least two batches of biomethane, at least a portion of the first batch distributed to the feedstock, at least a portion of the second batch distributed to the fuel, a distribution of the at least two batches of biomethane providing the feedstock with biomethane having first weighted average carbon intensity and the fuel with biomethane having a second weighted average carbon intensity, the first weighted average carbon intensity being different than the second weighted average carbon intensity.

In accordance with one aspect of the instant invention there is provided a method of producing fuel or a chemical product, the method comprising: (a) providing at least two batches of biomethane, the at least two batches of biomethanes including a first batch of a first biomethane and a second batch of a second biomethane, the first and second biomethanes being produced from different feedstocks, being produced from different processes, having different carbon intensities, or any combination thereof; the at least two batches of biomethane for use in a production process that includes hydrogen production, the hydrogen production comprising: (i) subjecting feedstock to methane reforming to produce syngas, (ii) combusting fuel for producing heat for the methane reforming, and (iii) subjecting the syngas or a gas derived from the syngas to a hydrogen purification process; and (b) generating a quantity of hydrogen from the hydrogen production using the at least two batches of biomethane, at least a portion of the first batch distributed to the feedstock, at least a portion of the second batch distributed to the fuel, a distribution of the at least two batches of biomethane distributing at least one of the at least two batches of biomethane disproportionally between the feedstock and the fuel.

### BRIEF DESCRIPTION OF THE DRAWINGS

Further features and advantages of the present disclosure will become apparent from the following detailed description, taken in combination with the appended drawings, in which:

FIG. 1a is a schematic diagram of hydrogen production based on SMR;

FIG. 1b is a schematic diagram of hydrogen production based on ATR;

FIG. 2a is a schematic diagram of hydrogen production based on SMR and an absorption-based hydrogen purification; and

FIG. 2b is a schematic diagram of hydrogen production based on SMR and an adsorption-based hydrogen purification with recycle of the tail-gas.

### DETAILED DESCRIPTION

### Biomethane

The method(s) of the present disclosure include providing at least two different biomethanes for producing hydrogen from a hydrogen production process. Each of these biomethanes is derived from biomass.

Biomass refers to organic material originating from plants, animals, or micro-organisms (e.g., including plants, agricultural crops or residues, municipal wastes, animal wastes, and algae). Biomass is a renewable resource, which can be naturally replenished on a human timescale, and which can be used to produce bioenergy and/or biofuels (e.g., biogas). In general, the biomass can be any suitable biomass (e.g., one or more types of biomass feedstock). Some examples of biomass from which biomethane can be derived include, but are not limited to: (i) energy crops (e.g., switchgrass, sorghum, etc.); (ii) residues, byproducts, or waste from the processing of plant material in a facility, or feedstock derived therefrom (e.g., sugarcane bagasse, sugarcane tops/leaves, corn stover, etc.); (iii) agricultural residues (e.g., wheat straw, corn cobs, barley straw, corn stover, etc.); (iv) forestry material; (v) livestock manure, such as sheep, swine, and cow manure; (vi) food scraps and/or agrifood processing residues (e.g., from slaughterhouse), (vii) municipal waste or components removed or derived from municipal waste, and/or (viii) wastewater sludge. In certain embodiments, the biomass is a fibrous biomass (e.g., straw). In certain embodiments, the biomass is waste material (e.g., manure).

Each biomethane is produced in a biomethane production process that converts biomass to biomethane (i.e., on a human timescale). Each biomethane production process can include any suitable process or combination of processes that converts the biomass to biomethane. For example, a biomethane production process can include anaerobic digestion of biomass (e.g., gaseous, liquid, or solid) followed by biogas upgrading. Alternatively, a biomethane production process can include a thermochemical conversion of biomass. Thermochemical conversion of biomass can include thermal gasification of biomass (e.g., woody biomass) followed by methanation, or can include a process based on pyrolysis of biomass.

In certain embodiments, at least one of the biomethanes is produced in a biomethane production process that includes anerobic digestion. In certain embodiments, all of the biomethanes are produced in a biomethane production process that includes anerobic digestion. Anaerobic digestion refers to the biological breakdown of organic matter by anaerobic microorganisms, is typically conducted in anaerobic or low oxygen conditions, and may involve a series of microorganism types and processes (e.g., hydrolysis, acidogenesis, acetogenesis, and methanogenesis). In general, the anaerobic digestion of biomass can be conducted in any suitable environment, including a natural environment (e.g., a landfill) or a controlled environment (e.g., one or more anaerobic digester reactors arranged in series and/or in parallel). Each anaerobic digester can be a holding tank, or another contained volume, such as a covered lagoon or sealed structure, configured to facilitate the anaerobic digestion and collection of biogas. For example, each anaerobic digester can be a plug flow system or basin type reactor. Such anaerobic digesters can be single-stage or multi-stage digester systems and/or may be designed and/or operated in a number of configurations including batch or continuous, mesophilic or thermophilic temperature ranges, mixed or unmixed, and low, medium, or high rates. The anaerobic digestion conducted in such digesters can use a nutrient solution, which may improve the conversion, particularly for fibrous biomass. Using a controlled environment facilitates monitoring input and output material flows.

Anaerobic digestion produces biogas, which is a gas mixture that typically contains methane (CH₄) and carbon dioxide (CO₂), and that may contain water (H₂O), nitrogen (N₂), hydrogen sulfide (H₂S), ammonia (NH₃), oxygen (O₂), volatile organic compounds (VOCs), and/or siloxanes, depending on the biomass from which it is produced. Biogas often has a methane content between about 35% and about 75% (e.g., about 60%) and a carbon dioxide content between about 15% and about 65% (e.g., about 35%). The percentages used to quantify gas composition and/or a specific gas content, as used herein, are expressed as mol %, unless otherwise specified. More specifically, they are expressed by mole fraction at standard temperature and pressure (STP), which is equivalent to volume fraction.

Biogas is often upgraded in a biogas upgrading process. Biogas upgrading refers to one or more processes where biogas (e.g., raw or cleaned biogas) is treated to remove one or more components (e.g., CO₂, N₂, H₂O, H₂S, O₂, NH₃, VOCs, siloxanes, and/or particulates), wherein the treatment increases the calorific value of the biogas. For example, biogas upgrading typically includes removing carbon dioxide and/or nitrogen (if present in significant amounts). In general, biogas upgrading can be conducted using any suitable technology or combination of technologies known in the art. Biogas upgrading, which is well-known, often includes one or more of the following technologies: 1) absorption, 2) adsorption, 3) membrane separations, and 4) cryogenic upgrading. As will be understood by those skilled in the art, the technology or combination of technologies selected may be dependent on the composition of the biogas and/or how it is produced. Since biogas often has a significant carbon dioxide content, biogas upgrading plants often include at least one system for separating methane from carbon dioxide. Some examples of technologies that can remove carbon dioxide from biogas include, but are not limited to, absorption (e.g., water scrubbing, organic physical scrubbing, chemical scrubbing (e.g., amine)), adsorption (e.g., pressure swing adsorption (PSA), which includes vacuum PSA, or temperature swing adsorption), membrane separation (e.g., CO₂ selective membranes based on polyimide, polysulfone, cellulose acetate, polydimethylsiloxane), and cryogenic separation.

In general, biogas upgrading can produce an upgraded biogas (e.g., biomethane). When conducted in one or more anaerobic digesters, the anaerobic digestion can also produce a potentially usable digestate. Digestate refers to the material remaining after one or more stages of anaerobic digestion (e.g., may refer to acidogenic digestate, methanogenic digestate, or a combination thereof). Digestate can include organic material not digested by the anaerobic microorganisms (e.g., fibrous undigested organic material made of lignin and cellulose), byproducts of the anaerobic digestion released by the microorganisms, and/or the microorganisms themselves. For example, the digestate can include carbohydrates, nutrients (such as nitrogen compounds and phosphates), other organics, and/or wild yeasts. The composition of digestate can vary depending on the biomass from which it is derived. Digestate often has both a solid and liquid component. One use of digestate is as a soil conditioner, where it can provide nutrients for plant growth and/or displace the use of fossil-based fertilizers. However, as a soil conditioner, digestate may have a significant methane formation potential, and thus may be associated with GHG emissions.

In certain embodiments, at least one of the biomethanes is produced in a biomethane production process that includes gasification followed by methanation. In certain embodiments, at least one of the biomethanes is produced in a biomethane production process that includes gasification followed by methanation, while at least one other of the biomethanes is produced in a biomethane production process that includes anaerobic digestion. Gasification refers to a process that converts biomass and/or fossil-based carbonaceous materials at high temperatures (e.g., >700° C.), without combustion, with a controlled amount of oxygen and/or steam into gas mixture primarily composed of carbon monoxide (CO) and hydrogen and sometimes carbon dioxide (i.e., syngas). For example, syngas produced by the gasification of wood may include carbon monoxide, carbon dioxide, hydrogen, methane, ethylene (C₂H₄), ethane (C₂H₆), dust (ash), tar, chloride, sulfur, etc. Following gasification, the syngas is often subjected to cooling, tar removal, and/or cleaning. The syngas may then be subjected to methanation, a catalytic conversion wherein carbon dioxide and carbon monoxide in the syngas can undergo the following reactions:

CO+3H₂→CH₄+H₂O (1)

CO₂+4H₂→CH₄+2H₂O (2)

Methanation, which is well-known in the art, typically is carried out in the presence of a solid catalysis (e.g., nickel-based catalyst). The gas produced by gasification followed by methanation typically contains methane (and possibly ethane) and water, and can include carbon dioxide. Methanation units, which can include water gas shift (WGS), carbon dioxide scrubbing, methanation, and dehydration, are often configured to produce biomethane. A possible byproduct of biomass gasification is biochar (biological charcoal).

In certain embodiments, one or more of the biomethane production process includes one or more processes for purifying a gas mixture containing methane derived from the biomass (e.g., biogas upgrading, methanation, etc.). In certain embodiments, gas having a relatively high calorific content (e.g., propane or natural gas) is blended with the biomethane in order to increase the calorific content thereof (e.g., the biomethane may be enriched as part of biogas upgrading). In general, each of the biomethanes produced will be of sufficient quality for it to be substantially interchangeable with natural gas (e.g., without or after being blended with propane). Biomethane is generally considered interchangeable with natural gas when it can be used without the need for any changes in transmission and distribution infrastructure and/or end-user equipment. For example, in certain embodiments, each of the biomethane production processes produces biomethane having a methane content of least about 90%, at least about 91%, at least about 92%, at least about 93%, at least about 94%, at least about 95%, at least about 96%, at least about 97%, at least about 98%, or at least about 99%.

In certain embodiments, one or more, or each of the biomethanes is of sufficient quality that it can be injected into a natural gas distribution for transport to hydrogen production (e.g., optionally after propane or natural gas is blended in). The term “distribution system”, as used herein, refers to a single pipeline or interconnected network of pipelines (i.e., physically connected). Distribution systems are used to distribute a product (e.g., natural gas, hydrogen, etc.), often to multiple users and/or destinations (e.g., businesses and households). A distribution system can include pipelines owned and/or operated by different entities and/or pipelines that cross state, provincial, and/or national borders, provided they are physically connected. One example of a distribution system is the U.S. natural gas grid, which includes interstate pipelines, intrastate pipelines, and/or pipelines owned by local distribution companies. When biomethane is transported by a natural gas distribution system, a quantity (e.g., in MJ) of biomethane produced is injected into the natural gas distribution system at one location and an equal quantity (e.g., in MJ) of gas is withdrawn from the natural gas distribution system at another location. Since the transfer or allocation of the environmental attributes of biomethane injected into a natural gas distribution system to gas withdrawn at a different location is typically recognized, the withdrawn gas is recognized as the injected biomethane and/or is treated as biomethane under applicable regulations (e.g., even though the withdrawn gas may not contain actual molecules from the original biomass and/or contains methane from fossil sources). Such transfer may be carried out on a displacement basis, where transactions within the natural gas distribution system involve a matching and balancing of inputs and outputs. Typically, the direction of the physical flow of gas is not considered. The term “environmental attributes”, as used herein with regard to a specific material (e.g., biomethane), refers to any and all attributes related to the material, including all rights, credits, benefits, or payments associated with the renewable nature of the material and/or the reduction in or avoidance of fossil fuel consumption or reduction in lifecycle GHG gas emissions associated with the use of the material. Some non-limiting examples of environmental attributes include verified emission reductions, voluntary emission reductions, offsets, allowances, credits, avoided compliance costs, emission rights and authorizations, certificates, tax benefits, voluntary carbon units, under any law or regulation, or any emission reduction registry, trading system, or reporting or reduction program for GHG gas emissions that is established, certified, maintained, or recognized by any international, governmental, or nongovernmental agency.

Although each biomethane production process produces biomethane (i.e., a gas at standard temperatures and pressures that is at least about 80% renewable methane), the term “biomethane”, as used herein, can also refer to natural gas withdrawn from a natural gas distribution system that is associated with the environmental attributes of biomethane injected into the natural gas distribution system (e.g., a gas that is treated as renewable under applicable regulations).

In certain embodiments, one or more, or each, of the biomethanes produced is of sufficient quality to (1) meet or exceed applicable natural gas distribution system specifications (e.g., pipeline specifications) or (2) meet or exceed applicable quality specifications for vehicle use (e.g., CNG specifications). Pipeline specifications, which can include specifications required for biogas for injection into a natural gas distribution system, may vary by region and/or country in terms of value and units. For example, pipelines standards may require the biomethane to have a CH₄ content that is at least about 95% or have a heating value of at least about 950 BTU/scf.

In general, each of the biomethanes can be produced close to hydrogen production or can be transported to hydrogen production. For example, biomethane can be compressed and transported by vehicle (e.g., as compressed natural gas (CNG)), can be liquified and transported by vehicle (e.g., depending on its composition), and/or can be transported via a natural gas distribution system (e.g., as a fungible batch).

As discussed herein, the method(s) of the present disclosure include providing at least two different biomethanes for producing hydrogen from a hydrogen production process. The phrase “different biomethanes”, as used herein, refers to biomethanes that differ from each other in terms of their source, the feedstock from which they are derived, their production processes, and/or their carbon intensity. The different biomethanes can be provided simultaneously, or separately (e.g., one after the other), as long as they are provided for producing a given quantity of hydrogen (e.g., a batch of hydrogen and/or an amount of hydrogen produced during a particular reporting period). For example, each batch of biomethane may be designated for use as feedstock and/or fuel for a given time period (e.g., reporting period).

In certain embodiments, at least two of the biomethanes differ from each other in terms of their source. The source of biomethane, which generally refers to the point of origin of the biomethane, can refer to the geographical location from which the biomethane originated and/or a system that converts biomass to a methane-based renewable gas (e.g., to raw biogas and/or biomethane). For example, some examples of biomethane sources include, but are not limited to, farms having one or more anaerobic digesters, landfills, and gasification plants. Each source of biomethane can produce a single biomethane having a single carbon intensity and/or that is derived from a single type of feedstock, or can produce two or more biomethanes, each of which can have a different carbon intensity and/or can be from a different feedstock. For example, an anaerobic digester can process feedstock containing manure and straw. In this case, the biomethane produced can be nominally divided into a first biomethane produced from a manure feedstock and a second biomethane produced from a straw feedstock, in a ratio determined at least in part by feedstock inputs. Although produced from the same source (e.g., anaerobic digester), the different biomethanes may have different carbon intensities and/or sustainability characteristics. In certain embodiments, at least two of the biomethanes differ from each other in terms of the country in which they originated.

In certain embodiments, at least two of the biomethanes differ from each other in terms of the feedstock from which they are derived. Biomethane derived from different feedstocks may qualify for different credits under certain regulations. For example, the United States Environmental Protection Agency (EPA) allows biomethane produced from landfill gas to qualify for cellulosic biofuel (D3) RINs, whereas biomethane derived from non-cellulosic feedstocks like fats, oils, sugars, starches, and most food wastes typically qualifies for D5 RINs. In addition, different types of feedstocks may have different sustainability characteristics. For example, energy crops (e.g., low-cost and low-maintenance crops grown solely for energy production rather than food) may be associated with potential land-use impacts and/or may require fertilizer produced from fossil fuels. The production of biomethane from feedstock that is based on waste such as livestock manure or food scraps can prevent methane from being released to the atmosphere that otherwise would escape to the atmosphere as it decomposed, and thus may be associated with avoided GHG emissions (e.g., may have negative carbon intensities). Sustainability characteristics may include, but are not limited to, the feedstock, country of origin of biomethane, carbon intensity of biomethane, and/or biomethane production process (e.g., see the International Sustainability & Carbon Certification (ISCC)).

In certain embodiments, at least two of the biomethanes differ from each other in terms of one or more of their sustainability characteristics. In certain embodiments, at least two of the biomethanes differ from each other in terms of the feedstock from which they are derived (e.g., energy crop versus agricultural residue). For example, in certain embodiments, one of the biomethanes is derived from a feedstock that is considered a waste and/or residue under applicable regulations, whereas another is derived from a feedstock that is not considered a waste or residue. In some jurisdictions, if a feedstock is categorized as waste and/or residue it may be double counted in terms of credits (e.g., under the UKs Renewable Transport Fuel Obligation (RTFO). In some instances, it can be advantageous to use biomethane produced from waste and/or residue as feedstock. Sustainability characteristics are well known in the art and can be readily determined by those of ordinary skill in the art (e.g., according to applicable regulations). When one or more sustainability characteristics of the biomethanes are being compared, the sustainability characteristics of the biomethane(s) in the fuel and feedstock are determined using the same methodology and/or applicable regulations. For example, in certain embodiments, at least two of the biomethanes differ from each other in terms of one or more sustainability characteristics recognized by the ISCC.

In certain embodiments, at least two of the biomethanes differ from each other in terms of the process that produces the biomethane. For example, in certain embodiments, at least two biomethanes differ from each other in terms of the type of biomethane production process (e.g., anerobic digestion or gasification). In certain embodiments, at least two of the biomethanes differ from each other in terms of the anaerobic digestion process (e.g., in one process digestate is fed to an open lagoon and in the other process digestate is fed to a closed lagoon). In certain embodiments, at least two of the biomethanes differ from each other in terms of the biogas upgrading (e.g., in one process off-gas from biogas upgrading is released to the atmosphere and in the other process off-gas from biogas upgrading is flared (e.g., thermal oxidation)). In certain embodiments, at least two of the biomethanes differ from each other in terms of the whether renewable power is used in the process. In certain embodiments, at least two of the biomethanes differ from each other in terms of whether co-products are produced during biomethane production (e.g., carbon dioxide captured and provided for sequestration). In some instances, it can be advantageous to use biomethane produced in processes that are associated with a relatively low carbon intensity (e.g., covered lagoons, combusting off-gas from hydrogen purification, using renewable power, providing CCS) as fuel for methane reforming.

In certain embodiments, at least two of the biomethanes differ from each other in terms of their carbon intensity and/or lifecycle greenhouse gas (GHG) emissions. The term “carbon intensity” or “CI” refers to the quantity of lifecycle GHG emissions associated with a product (e.g., fuel) for a given production process and is often expressed in grams of CO₂ equivalent emissions per unit of product produced (e.g., gCO₂e/MJ of fuel, gCO₂e/MMBTU of fuel, gCO₂e/kWh of electricity, or kgCO₂e/kg of fuel/product). As will be understood by those skilled in the art, lifecycle GHG emissions and/or carbon intensity are often determined using a Lifecycle Analysis (LCA), which identifies and estimates all GHG emissions in producing a fuel/product, from the growing or extraction of raw materials, to the production of the fuel/product, through to the end use (e.g., well-to-wheel). For example, the carbon intensity and/or lifecycle GHG emissions of each biomethane can account for carbon emissions associated with feedstock production (e.g., fertilizer use), biogas upgrading (e.g., compression), and/or transport via natural gas pipeline (e.g., methane losses). In addition, the carbon intensity and/or lifecycle GHG emission of each biomethane can account for carbon emission credits, such as those associated with feedstock production (e.g., using a waste feedstock associated with avoided GHG emissions) and/or biomethane production (e.g., CCS of carbon-containing material produced from biomethane production that is not converted to the biomethane). With regard to the latter, one or more carbon-containing material(s) derived from digestate, char, or carbon dioxide (e.g., produced during anaerobic digestion) can be stored as part of a CCS process, thereby reducing the carbon intensity of the biomethane. Those skilled in the art will understand that lifecycle GHG emissions and/or carbon intensity of a given fuel, fuel intermediate, or chemical product, can be dependent upon the LCA methodology used. For purposes herein, the carbon intensity of the biomethanes being compared (e.g., the first and second batches) will be determined using the same methodology.

In general, any methodology can be used to determine carbon intensity and/or lifecycle GHG emissions (e.g., of the biomethanes, hydrogen produced using the biomethanes, and/or fuel/product produced using the hydrogen). However, when the hydrogen and/or fuel/product produced using the hydrogen is treated as meeting a certain lifecycle GHG reduction threshold under certain regulations (e.g., is treated as clean or low carbon intensity hydrogen) and/or when the method includes obtaining one or more credits for the hydrogen and/or its production, and/or for fuel, fuel intermediate, or product produced from the hydrogen, or its production, the methodology will be selected to comply with the prevailing rules and regulations in the applicable jurisdiction (e.g., relevant to desired credits). Methodologies for calculating carbon intensities and/or lifecycle GHG emissions according to various regulatory bodies are well known in the art and can be readily calculated by those of ordinary skill in the art.

In certain embodiments, the carbon intensities and/or lifecycle GHG emissions are determined using a LCA model, such as the GREET model. The GREET model, which is well-known by those skilled in the art, refers to “The Greenhouse gases, Regulated Emissions, and Energy use in Technologies Model” developed at Argonne National Laboratory (ANL) (e.g., greet.es.anl.gov). In certain embodiments, the carbon intensities and/or lifecycle GHG emissions are determined based on the fuel/product being produced according to a certain pathway (e.g., a fuel pathway). For example, in certain embodiments, the carbon intensities are pathway certified carbon intensities or are regulatory default value carbon intensities. In general, the term “fuel pathway” refers to a collective set of processes, operations, parameters, conditions, locations, and technologies throughout all stages that the applicable agency considers appropriate to account for in the system boundary of a complete analysis of that fuel's lifecycle greenhouse gas emissions. In some cases, a fuel pathway can be specific combination of three components, namely: (1) feedstock, (2) production process, and (3) product or fuel type. In certain embodiments, the carbon intensities are regulatory default value carbon intensities. For example, in the UK, biomethane produced from wet manure may have a default carbon intensity of 22 gCO₂eq/MJ when the digestate is fed to an open enclosure, and when the off-gas from biogas upgrading is not combusted, or may have a default carbon intensity of −100 gCO₂eq/MJ when the digestate is fed to closed enclosure, and when the off-gas from biogas upgrading is combusted. In certain embodiments, the carbon intensities (e.g., of the biomethanes) are determined using disaggregated default values (e.g., associated with certain feedstocks and/or steps in a supply chain) or a mixture of disaggregated default values and measured values (e.g., based on supply chain specific measured values). In certain embodiments, the carbon intensities (e.g., of the biomethanes) are determined (e.g., using an LCA) and then verified by the regulatory agency (e.g., the fuel pathway and/or corresponding carbon intensities can be approved by the regulatory agency) and/or by a verification body approved and/or appointed by the regulatory agency. As discussed herein, the method(s) of the present disclosure include providing at least two different biomethanes for producing hydrogen from a hydrogen production process. In certain embodiments, at least one of these biomethanes is a consignment that is an aggregate of biomethanes having the same declared sustainability characteristics. For example, the carbon intensity of the consignment may be a regulatory default value carbon intensity. In certain embodiments, at least one of the different biomethanes is processed at a central biogas upgrading facility that receives biogas from multiple sources, and which may produce one or more biomethanes.

Advantageously, in providing at least two different biomethanes (e.g., having different carbon intensities, lifecycle GHG emissions, and/or sustainability characteristics), the relative amount of each biomethane provided for producing a certain quantity of hydrogen can be tailored (e.g., to reduce cost and/or accommodate supply). For example, biomethane having a relatively low carbon intensity (e.g., manure-based biomethane) may be provided in a smaller amount than biomethane having a relatively high carbon intensity (e.g., landfill gas).

### Hydrogen Production

The method(s) of the present disclosure include providing at least two different biomethanes for producing hydrogen from a hydrogen production process. In general, the hydrogen production can use any suitable technology known in the art that can convert a methane-containing feed (e.g., containing biomethane and/or natural gas) to hydrogen. Examples of technologies that may be suitable for use in hydrogen production include, but are not limited to, steam methane reforming (SMR), autothermal reforming (ATR), partial oxidation (POX), and/or dry methane reforming (DMR). SMR, ATR, and DMR, which are types of catalytic reforming, may operate by exposing natural gas to a catalyst at high temperature and pressure to produce syngas. POX reactions, which include thermal partial oxidation reactions (TPOX) and catalytic partial oxidation reactions (CPOX), may occur when a sub-stoichiometric fuel-oxygen mixture is partially combusted in a reformer. POX also may be referred to as oxidative reforming. For purposes herein, the term “methane reforming” may refer to reforming based on SMR, ATR, DMR, and/or POX. Methane reforming is well known in art. Of the various types of methane reforming, SMR may be the most common.

In certain embodiments, the hydrogen production includes SMR. In SMR, which is an endothermic process, methane is reacted with steam under pressure in the presence of a catalyst to produce carbon monoxide (CO) and H₂ according to the following reaction:

CH₄+H₂O+heat→CO+3H₂ (3)

The SMR reaction typically occurs in SMR reactor tubes, which contain the reforming catalyst. Without being limiting, the catalyst may be nickel-based, the operating pressure may be between 200 psig (1.38 MPa) and 600 psig (4.14 MPa), and the operating temperature may be between about 450 to about 1000° C. The heat required for the steam methane reforming can be provided, at least in part, by combusting fuel provided to the reformer burners (e.g., a combustion chamber typically surrounds the reformer tubes that contains the catalyst and in which the reforming reaction is conducted). The reformers may be characterized by the location of the burners within the combustion chamber (e.g., side-fired, top-fired, bottom-fired).

The syngas produced from SMR may be further reacted in a water gas shift (WGS) reaction, wherein carbon monoxide is converted to carbon dioxide and hydrogen:

CO+H₂O→CO₂+H₂+small amount of heat (4)

While the catalyst provided for the SMR reaction discussed with regard to Eq. 3 may be active with respect to the WGS reaction in Eq. 4 (e.g., the gas leaving the steam reformer may be in equilibrium with respect to the WGS reaction), one or more WGS reactors are often provided downstream of one or more SMR reactors. In general, WGS may use any suitable type of shift technology (e.g., high temperature shift conversion, medium temperature shift conversion, low temperature shift conversion, sour gas shift conversion, or isothermal shift). For example, WGS reactions may be conducted at temperatures between 320-450° C. (high temperature) and/or between 200-250° C. (low temperature). Without being limiting, high temperature WGS may be conducted with an iron oxide catalyst (e.g., supported by chromium oxide), whereas low temperature WGS may be conducted with a Cu/ZnO mixed catalyst. Optionally, a promoter can be added. In general, there may be one or more stages of WGS. For example, the WGS may be conducted in a high temperature WGS reactor (e.g., 350° C.) followed by a low temperature WGS reactor (e.g., 200° C.).

Although optional, providing WGS downstream of methane reforming increases the yield of H₂, and thus is commonly included in hydrogen production. When included, the WGS is considered to be part of the methane reforming. The syngas produced from methane reforming often includes hydrogen, methane, carbon monoxide, carbon dioxide and water vapour. Without being limiting, the syngas from the methane reforming (e.g., which may be at about 210 to about 220° C.) can be cooled (e.g., to about 35 to about 40° C.), and the condensate separated, prior to hydrogen purification.

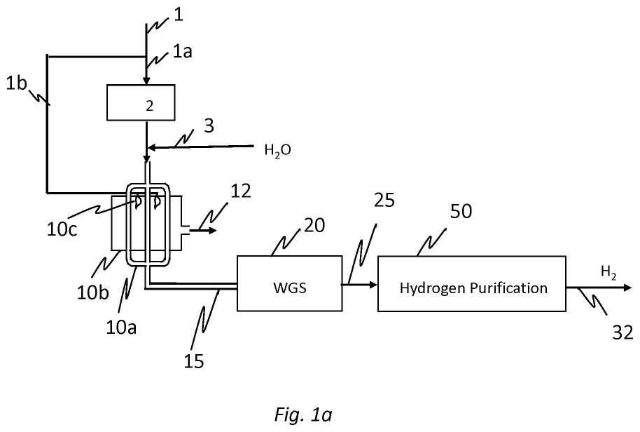

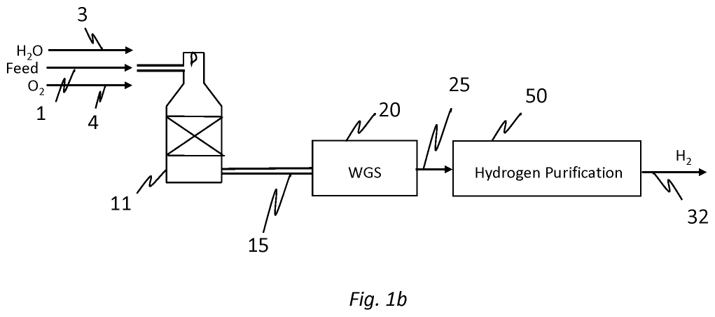

FIG. 1a illustrates an embodiment of hydrogen production based on SMR. A feed 1 (e.g., natural gas) is split into a first portion 1a and a second portion 1b. The first portion 1a is subjected to an optional desulfurization 2 (e.g., depending on its composition) and is fed along with steam 3 into the reactor tubes 10a of the steam methane reformer, which contain the reforming catalyst. The second portion 1b is fed into the combustion chamber 10b that surrounds the reactor tubes 10a and that is equipped with reformer burners 10c. Combustion of this fuel 1b provides at least some of the heat required for the steam methane reforming of the first portion 1a. The reaction products from the combustion, which typically includes carbon dioxide, can be fed to a stack and released as flue gas 12. The syngas 15 produced from the SMR is fed to WGS 20 to produce more hydrogen. The resulting syngas 25, or shifted gas, is fed to hydrogen purification 50 to produce a gas enriched in hydrogen 32. Optionally, the first portion 1a is subjected to pre-reforming (not shown) prior to being fed into the reforming tubes.

In certain embodiments, the hydrogen production includes ATR. In contrast to SMR, wherein the catalyst is contained in tubes that are heated by an external burner, in ATR a first portion of the feed is burned in the reactor to raise the temperature of the remaining feed before it contacts the catalyst provided in a different region of the same reactor. ATR may be viewed as a combination of partial oxidation and catalytic steam reforming (e.g., Eq. 5) or as a combination of partial oxidation and carbon dioxide reforming (e.g., Eq. 6). Since the heat generated from the partial oxidation (e.g., in the combustion zone of the reactor) may be used in the methane reforming (e.g., in the reforming zone of the reactor), a stand-alone ATR may not require the supply or dissipation of thermal energy.

4CH₄+O₂+2H₂O→10H₂+4CO (5)

2CH₄+O₂+CO₂→3H₂+3CO+H₂O (6)

Without being limiting, conventional ATR may operate at temperatures between about 750 to about 1400° C. ATR, which is often used for smaller scale hydrogen production, may afford higher hydrogen production than POX and faster start-up and response times than SMR.

FIG. 1b illustrates an embodiment of hydrogen production based on ATR. Referring to FIG. 1b, the feed 1 (e.g., natural gas) is fed, along with steam 3 and oxygen 4, into the ATR reactor 11. The reactor 11 has an upper region having one or more burners and lower region containing the catalyst. Combustion of a portion of the feed stream from the burners in the upper region provides at least some of the heat required for the endothermic reaction occurring in the lower region. The syngas 15 produced is fed to WGS 20 to produce more hydrogen. The resulting syngas 25, or shifted gas, is fed to hydrogen purification 50 to produce a gas enriched in hydrogen. Optionally, the feed 1 is subjected to desulfurization and/or pre-reforming (not shown) prior to being fed into the reactor 11.

In certain embodiments, the hydrogen production includes DMR. In contrast to SMR, DMR reacts the methane with carbon dioxide instead of water according to the following reaction:

CO₂+CH₄→2CO+2H₂ (7)

Without being limiting, the DMR catalyst may be iron, ruthenium, palladium, or platinum based.

As will be understood by those skilled in the art, any methane reforming technology or combination of technologies can be used. For example, methane reforming units often include one or more reactors (e.g., one or more SMR reactors connected in series with one or more ATR reactors, or one or more ATR reactors connected in series with one or more WGS reactors). The above-described methods of converting a methane containing gas to hydrogen (e.g., using SMR, ATR, and/or DMR) are well-known in the art, and those in the art will understand that the methane reforming method, operating conditions, and/or configuration may be selected in dependence upon the feed and/or the desired product. For example, it may be advantageous to provide a purification stage and/or a pre-reforming stage for processing the feed upstream of the methane reforming. A purification stage may remove sulfur, chloride, olefin, and/or other compounds that may be detrimental to downstream reforming catalysts (e.g., SMR catalysts). Pre-reforming may allow a higher inlet feed temperature with minimal risk of carbon deposition. As will be understood by those skilled in the art, methane reformers are often configured to receive natural gas feeds (e.g., pipeline natural gas). The term “natural gas” or “NG”, as used herein, refers to mixture of hydrocarbon compounds that is gaseous at standard temperatures and pressures, where the primary component is methane. In general, the methane reformers in a hydrogen plant may be able to convert any of the hydrocarbons present in the natural gas to syngas (i.e., not just the methane). In certain embodiments, the feed for methane reforming contains natural gas, biomethane, refinery gas, liquid petroleum gas (LPG), and/or light naphtha.

In addition to methane reforming, the hydrogen production includes hydrogen purification. In hydrogen purification, the syngas (e.g., shifted gas) produced from methane reforming is subjected to processing wherein hydrogen is separated from carbon monoxide, carbon dioxide, and/or methane in one or more stages to produce a stream enriched in hydrogen (i.e., containing at least about 80% hydrogen). For example, in certain embodiments, the hydrogen purification produces a stream enriched in hydrogen having a hydrogen content of at least about 90%, about 92%, about 94%, about 96%, about 98%, about 99%, or about 99.5%. In certain embodiments, the hydrogen purification produces a stream enriched in hydrogen having a hydrogen content of at least about 99.9%. Without being limiting, some examples of suitable hydrogen purification technologies include, but are not limited to: a) absorption, b) adsorption, c) membrane separation, d) cryogenic separation, and/or e) methanation. Some examples of absorption systems that may be suitable include, but are not limited to, a monoethanolamine (MEA) unit or a methyldiethanolamine (MDEA) unit. A MEA unit may include one or more absorption columns containing an aqueous solution of MEA at about 30 wt %. The outlet liquid stream of solvent may be treated to regenerate the MEA and separate carbon dioxide. Some examples of adsorption systems that may be suitable include, but are not limited to, systems that use adsorbent bed (e.g., molecular sieves, activated carbon, active alumina, or silica gel) to remove impurities such as methane, carbon dioxide, carbon monoxide, nitrogen, and/or water from the syngas. Methanation is a catalytic process that can be conducted to convert the residual carbon monoxide and/or carbon dioxide in the syngas to methane. For example, see Eqs. 1 and 2. Since the methanation reaction consumes hydrogen, a hydrogen purification unit that includes methanation may include carbon dioxide removal prior to methanation.

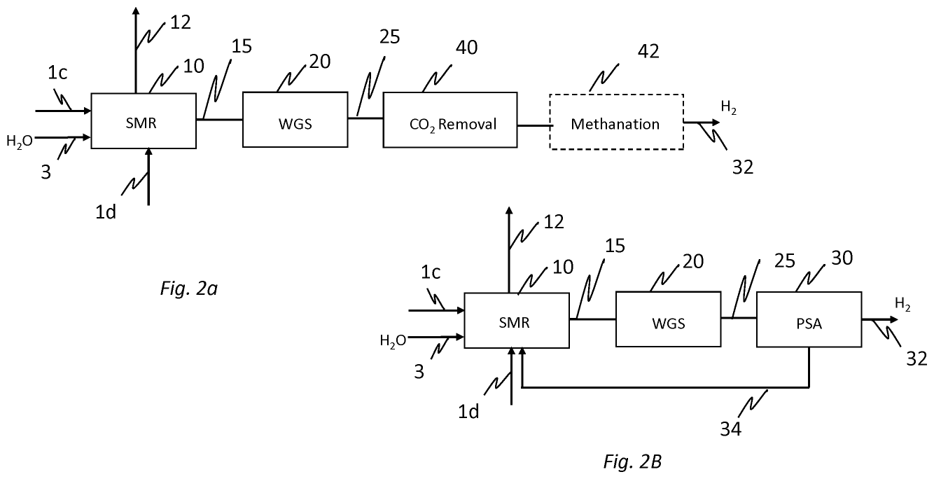

Referring to FIG. 2a, there is shown an embodiment of hydrogen production wherein hydrogen purification is based on absorption. Feedstock 1c is fed, along with steam 3, into the reactor tubes used for SMR 10, which contain the reforming catalyst. Fuel Id is fed into the SMR burners, which provide the heat required for the endothermic reforming. The syngas 15 produced from the SMR 10 is fed to WGS 20 to produce more hydrogen. The resulting syngas 25, which may also be referred to as shifted gas, is cooled (not shown) and subjected to an absorption-based hydrogen purification. The absorption-based hydrogen purification includes a wet scrubbing carbon dioxide removal process 40 (e.g., amine absorption and regeneration cycle), and optionally includes a methanation process 42 to convert any remaining carbon monoxide and/or carbon dioxide to methane.

Referring to FIG. 2b, there is shown an embodiment of hydrogen production wherein hydrogen purification is based on adsorption. Feedstock 1c is fed, along with steam 3, into the reactor tubes used for SMR 10, which contain the reforming catalyst. Fuel Id is fed into the SMR burners, which provide the heat required for the endothermic reforming. The syngas 15 produced from the SMR 10 is fed to WGS 20 to produce more hydrogen. The resulting syngas 25, which may also be referred to as shifted gas, is cooled (not shown) and subjected to an adsorption-based hydrogen purification. The adsorption-based hydrogen purification includes pressure swing adsorption (PSA) 30. The PSA 30 produces a stream enriched in hydrogen 32 and tail gas 34. The tail gas 34, which may contain unconverted methane, hydrogen, carbon dioxide, and/or carbon monoxide, is fed back to SMR 10, where it is used to provide additional process heat for the SMR (e.g., fuel the SMR burners). More specifically, the tail gas 34 is combusted together with the fuel id. The use of PSA, and more specifically, the recycling of the tail gas to fuel the SMR burners, is generally associated with improved energy efficiency as less fuel id is required.

In various embodiments of the instant disclosure, the hydrogen production includes:
(i) subjecting feedstock to methane reforming to produce syngas,
(ii) combusting fuel for producing heat for the methane reforming, and
(iii) subjecting the syngas or a gas derived from the syngas to a hydrogen purification process.

In general, the feedstock and fuel can be provided in the same stream or different streams. In certain embodiments, the feedstock and fuel are provided in different streams. For example, referring to FIG. 1a, the first portion 1a and the second portion 1b, which were split from the same feed 1, are provided in separate streams, each of which is directed to a different area of the reformer (i.e., the two areas are physically separated). In this embodiment, some or all of the first portion 1a is feedstock for the hydrogen production. The term “feedstock”, as used herein, refers to material entering a process that contributes atoms to any product of the process or is deemed to contribute atoms to any product (e.g., hydrogen). Alternatively, the feedstock (e.g., 1c) and fuel (e.g., 1d) can be provided from separate sources (e.g., one being a natural gas distribution system and the other being one or more vessels transported by vehicle). In certain embodiments, at least a portion of each of the feedstock and fuel are provided in the same stream. For example, referring to FIG. 1b, although the feed 1 is not physically separated into two streams prior to entering the reactor, it can be nominally divided into a first portion that is combusted, and thus is fuel for providing heat for the methane reforming, and a second other portion that is converted to at least hydrogen and carbon monoxide (and possibly carbon dioxide), and thus is feedstock for the methane reforming. Referring to FIG. 2b, the feed 1c can be nominally dived into a first portion that is converted to at least hydrogen and thus is feedstock, and a second portion that passes through the steam reformer unconverted and is recycled to the burners as part of the tail gas 34, and thus is fuel for producing heat for the methane reformer.

In certain embodiments, the syngas produced from methane reforming (e.g., output from WGS) is fed directly to hydrogen purification. In certain other embodiments, the syngas produced from methane reforming (e.g., output from WGS) is processed prior to hydrogen purification. For example, in certain embodiments, carbon dioxide from the syngas is removed prior to, as part of, or subsequent to hydrogen purification and provided for storage as part of a CCS process.

In general, each of the feedstock and/or fuel can include only biomethane and/or can include one or more biomethanes in addition to any other suitable feedstocks/fuels (e.g., non-renewable feedstock/fuel). For example, natural gas withdrawn from a natural gas distribution system is commonly used as feedstock and/or fuel for hydrogen production. Alternatively, or additionally, the feedstock and/or fuel can include refinery gas, which generally refers to the lightest hydrocarbon stream produced from refinery process units and is typically made of methane and ethane and some other components. It can be particularly advantageous to provide feedstock and/or fuel that combines biomethane with non-renewable methane based gases. In certain embodiments, the feedstock includes one or more biomethanes and non-renewable natural gas and/or refinery gas. In certain embodiments, the fuel includes one or more biomethanes and non-renewable natural gas and/or refinery gas. In certain embodiments, each of the feedstock and fuel includes one or more biomethanes and non-renewable natural gas and/or refinery gas.

In general, each of the multiple biomethanes can be provided as a separate batch. The term “providing”, as used herein with respect to an element, refers to directly or indirectly obtaining the element and/or making the element available for use. The term “batch”, as used herein with respect to a gas (e.g., gas mixture), refers to a certain amount of the gas (e.g., measured using volume, mass, and/or energy delivered) and does not imply or exclude an interruption in the production and/or delivery. For example, a batch of biomethane can refer to biomethane withdrawn from a natural gas distribution at a certain rate (e.g., MJ/hour for a certain time period), can refer to a quantity of biomethane withdrawn from one or more CNG trailers, or can refer to a consignment of biomethane withdrawn from a natural gas distribution system (e.g., obtained via a book-and-claim process).

In general, the different batches of biomethane are provided to produce a quantity of hydrogen (e.g., hydrogen produced over a certain time period). The different batches of biomethane can be provided simultaneously, or separately (e.g., one after the other), as long as they are associated with producing the given quantity of hydrogen (e.g., a batch of hydrogen and/or an amount of hydrogen produced during a particular reporting period).

### Distribution of the Biomethanes

The method(s) of the present disclosure generally include distributing each of the multiple biomethanes between feedstock and fuel for the hydrogen production. Distributing a batch of biomethane between feedstock and fuel can refer to physically directing a certain share (e.g., about 0% to about 100%) of the batch such that it is provided as fuel while any remaining share is provided as feedstock, or can refer to allocating a certain share (e.g., about 0% to about 100%) of the batch such that it is designated as fuel while any remaining share is designated as feedstock (e.g., on paper and/or electronically).

As described herein, it has now been recognized that it can be advantageous to distribute the multiple biomethanes between the feedstock and fuel such that biomethane in the feedstock for methane reforming and biomethane in the fuel for methane reforming have different fractional make-ups and/or such that at least a portion of one of the biomethanes is feedstock for methane reforming and at least a portion of another of the biomethanes is fuel for methane reforming.

For purposes herein, the term “fractional make-up” refers to the composition in terms of the energy fractions of the constituent biomethanes. Accordingly, different fractional make-ups can result when at least one of the biomethanes is distributed disproportionally such that its energy fraction in the fuel for methane reforming is different from its energy fraction in the feedstock for methane reforming. The energy fraction of a given biomethane in the fuel refers to the energy of the given biomethane in the fuel in MJ divided by the sum of the energy of all biomethanes in the fuel in MJ. The energy fraction of a given biomethane in the feedstock refers to the energy of the given biomethane in the feedstock in MJ divided by the sum of the energy of all biomethanes in the feedstock in MJ. For purposes herein, an energy fraction can be 0, 1, or any value therebetween, and/or may be expressed as a percentage. For example, in some cases, the fractional make-up of the feedstock or fuel is about 100% of a given biomethane.

In certain embodiments of the disclosure, the multiple biomethanes are distributed such that biomethane in the feedstock has a first fractional make-up and biomethane in the fuel has a second fractional make-up, where the first and second fractional make-ups are different. For example, consider the following. If the distribution provides the feedstock for methane reforming with biomethane that includes 40 MJ of a first biomethane and 60 MJ of a second biomethane, and the fuel for methane reforming with biomethane that includes 60 MJ of the first biomethane and 40 MJ of the second biomethane, then the fractional make-up of the feedstock (40% of a first biomethane; 60% of a second biomethane) will be different from the fractional make-up of the fuel (60% of the first biomethane; 40% of the second biomethane). In contrast, if the distribution provides the feedstock for methane reforming with biomethane that includes 40 MJ of a first biomethane and 60 MJ of a second biomethane, and the fuel for methane reforming with biomethane that includes 20 MJ of the first biomethane and 30 MJ of the second biomethane, then the fractional make-up of the feedstock (40% of a first biomethane; 60% of a second biomethane) will be the same as the fractional make-up of the fuel (40% of the first biomethane; 60% of the second biomethane). In certain embodiments, the feedstock and/or fuel will contain non-renewable gas (e.g., refinery gas or natural gas) in addition to one or more biomethanes. In such embodiments, the amount of non-renewable gas is not factored in when determining the fractional make-up of biomethane in the feedstock and/or fuel (i.e., the fractional make-up of biomethane in the feedstock is determined relative to the total amount of biomethane in the feedstock, not relative to the total amount of gas in the feedstock).

In certain embodiments of the disclosure, at least one of the batches of biomethane is distributed disproportionally between the feedstock for methane reforming and the fuel for methane reforming (e.g., such that biomethane in the feedstock and biomethane in the fuel have different fractional make-ups). In a disproportional distribution of a given biomethane, the given biomethane is distributed such that its energy fraction in the fuel for methane reforming is different from its energy fraction in the feedstock for methane reforming. In contrast, in a proportional distribution of a given batch of biomethane, the energy fraction of the given biomethane is the same fraction in the fuel as in the feedstock. For example, consider the case where a first batch containing 30 MJ of a first biomethane and second batch containing 100 MJ of a second biomethane are provided for methane reforming. The energy fraction of the first biomethane relative to the total amount of biomethane provided is 0.23. A proportional distribution of the first biomethane results in the energy fraction of the first biomethane being 0.23 in each of the feedstock and the fuel. If only the two batches of biomethane are used as feed for methane reforming, and if 90% of the feed is feedstock, and 10% is fuel, then this proportional distribution results in the feedstock (i.e., 117 MJ, which is 90% of the total feed) including about 27 MJ of the first biomethane and about 90 MJ of the second biomethane, while the fuel (i.e., 13 MJ, which is 10% of the total feed) includes about 3 MJ of the first biomethane and about 10 MJ of the second biomethane. In general, a proportional distribution of each biomethane results when there is no selective distribution of the biomethane.

Embodiments that distribute at least one of the biomethanes disproportionally between fuel and feedstock, and/or distribute the multiple biomethanes such that biomethane in the fuel and feedstock have different fractional make-ups, are advantageous. For example, they allow the various characteristics of the biomethanes to be used beneficially, grouped beneficially, and/or averaged beneficially. In certain embodiments, distributing at least one of the biomethanes disproportionally between fuel and feedstock, and/or distributing the multiple biomethanes such that the biomethane in fuel and feedstock have different fractional make-ups, can provide the feedstock with certain sustainability characteristics, feedstock identity, and/or carbon intensity, while the fuel is provided with certain other sustainability characteristics, feedstock identity, and/or carbon intensity. In certain embodiments, the distribution provides the feedstock with at least 2, at least 3, or at least 4 different biomethanes. In certain embodiments, the distribution provides the fuel with at least 2, at least 3, or at least 4 different biomethanes. In certain embodiments, the distribution provides the feedstock with at least 2, at least 3, or at least 4 different biomethanes. In certain embodiments, the distribution provides each of the feedstock and the fuel with at least 2, at least 3, or at least 4 different biomethanes. In certain embodiments, at least 3, at least 4, or at least 5 different biomethanes are provided for methane reforming (e.g., each for use as feedstock and/or fuel).

In certain embodiments of the disclosure, the multiple batches of biomethane are distributed disproportionally between fuel and feedstock such that at least about 55%, at least about 60%, at least about 65%, at least about 70%, at least about 75%, at least about 80%, at least about 85%, at least about 90%, at least about 95%, or at least about 100% of the first batch of biomethane is provided for use as feedstock for the steam methane and at least about 55%, at least about 60%, at least about 65%, at least about 70%, at least about 75%, at least about 80%, at least about 85%, at least about 90%, at least about 95%, or at least about 100% of the second batch of a second biomethane is provided for use as fuel for producing heat for the methane reforming.

In certain embodiments, at least one of the biomethanes is distributed disproportionally between the fuel and feedstock such that each batch of biomethane is generally associated with either feedstock or fuel. For example, a batch of a first biomethane can be only associated with fuel, while a batch of second other biomethane can be only associated with feedstock. Providing one of the biomethanes largely for feedstock and another of the biomethanes largely for fuel, can allow each batch of the different biomethanes to be used for the most benefit, particularly when the feed for the hydrogen production also includes non-renewable natural gas. For example, consider the following.

While the use of one or more biomethanes can reduce the carbon intensity of hydrogen produced, where and/or how they are used (e.g., as feedstock and/or fuel) may determine the carbon intensity of the hydrogen produced, eligibility for fuel credits, and/or treatment under incentive programs (e.g., depending on prevailing regulations). A reason for this is that when multiple feedstocks (e.g., one or more biomethanes and/or natural gas) are used as feedstock for methane reforming, each batch of feedstock may be deemed to produce a different batch of hydrogen. For example, the co-processing of a feed comprising a biomethane feedstock and a natural gas feedstock can produce a first batch of hydrogen having a carbon intensity dependent on the biomethane feedstock and a second batch of hydrogen having a carbon intensity dependent on the natural gas feedstock. In contrast, when multiple feedstocks (e.g., one or more biomethanes and/or natural gas) are used as fuel for the methane reforming, the fuel may be treated as one gas (e.g., a having one carbon intensity).

In addition, while biomethane generally can be transported via a natural gas distribution system as a fungible batch, and thus withdrawn from the natural gas distribution system for use as feedstock or fuel, there is a possibility that under some regulations any renewable fuel used to produce heat for methane reforming may be considered a utility and/or may need to be physically transported to the hydrogen production as a segregated batch in order for the reduction in carbon intensity to be recognized by the prevailing regulatory authority (e.g., similar in concept to the behind-the-meter approach used for electricity).

Distributing at least one of the biomethanes disproportionally between feedstock and fuel, can allow each batch of the different biomethanes to be used for the most benefit. As described herein, different biomethanes can have different characteristics. For example, biomethane produced from landfill gas is often available in large quantities, but can have significant carbon emissions (e.g., a CI greater than 30 gCO₂eq/MJ). In contrast, biomethane produced from manure can have relatively low carbon emissions (e.g., a CI as low as −100 to −250 gCO₂eq/MJ), but may be relatively expensive and may be only available in smaller quantities. It can be advantageous to distribute the different biomethanes so that their characteristics (e.g., carbon intensity) can be used for the most benefit.

In certain embodiments, each batch of biomethane is distributed such that it is largely provided for use as feedstock or fuel. For example, it can be advantageous to distribute a batch of biomethane produced from landfill gas for use as feedstock, because it may be available in larger quantities and thus can increase the renewable content of the hydrogen provided (i.e., increase the yield of renewable hydrogen). It can be advantageous to distribute a batch of biomethane produced from manure for use as fuel, because even if it is only available in small quantities it can have a significant effect on the carbon intensity of the hydrogen produced even when used in small quantities and/or blended with non-renewable natural gas. For example, when a given biomethane is designated as fuel for the reforming, its carbon emissions can affect the carbon intensity of all of the hydrogen produced, even when the hydrogen is produced from different feedstocks. In contrast, when the same biomethane is designated as feedstock, its carbon emissions may be only associated with hydrogen produced from that feedstock. It may be advantageous to provide biomethanes having low and in particular negative carbon intensities to the fuel, particularly when the fuel contains another biomethane and/or natural gas, so that it can contribute to the weight averaged carbon intensity of the fuel and thus determine the carbon intensity of hydrogen, or fuel/product produced from the hydrogen, produced from all feedstocks for hydrogen production. In addition, such distributions can exploit different batches of biomethane being transported to hydrogen production by different methods. For example, biomethane produced from landfill gas is often produced in close proximity to established injection sites on a natural gas pipeline, whereas biomethane produced from manure-based biogas may not be in close proximity to an established injection site and it may be more efficient to transport it to hydrogen production by vehicle. Transporting biomethane produced from landfill gas via a natural distribution system and withdrawing it for use as feedstock for methane reforming, and transporting biomethane produced from waste or manure-based biogas by vehicle and providing it for use as fuel for the methane reforming is advantageous for various reasons, including but not limited to: 1) the biomethane feedstock withdrawn from the natural gas distribution system will have the same composition as withdrawn natural gas feedstock, and thus can be used interchangeable and/or in relatively large amounts for the methane reforming; 2) biomethane transported by vehicle for use as fuel does not necessarily have to meet or exceed pipeline quality, 3) costs may be reasonable to transport smaller amounts of biomethane by vehicle, and 4) in some cases, the use of biomethane as fuel to reduce the carbon intensity of a process is only recognized when the biomethane is physically generated on-site or physically transported to the site (i.e., as a segregated batch, as opposed to a fungible batch). In contrast, the use of biomethane withdrawn from a natural gas distribution system may comply with applicable regulations when the biomethane is used as a feedstock.

In certain embodiments, one of the batches of biomethane having the largest quantity on an energy basis is predominately provided for use as feedstock, while one of the batches having the smallest quantity on an energy basis is predominately provided for use as the fuel. In addition to exploiting the relatively large feedstock to fuel ratio often associated with hydrogen production (e.g., 70%:30%, or 90%:10%, or higher), this can produce at least one relatively large batch of renewable hydrogen (e.g., can increase the yield of renewable content). The term “renewable content”, as used herein, refers to the portion of the fuel(s) that is recognized and/or qualifies as renewable (e.g., a biofuel) under applicable regulations. The quantification of the renewable content can be determined using any suitable method and is typically dependent upon the applicable regulations.

In certain embodiments, one of the batches of biomethane having the largest quantity (based on energy) is predominately provided for use as feedstock, while one of the batches having the lowest carbon intensity is predominately provided for use as the fuel. Advantageously, this can provide the largest batch of renewable hydrogen, and optionally one or more batches of hydrogen generated from a non-renewable feedstock, with a relatively low carbon intensity.

In certain embodiments, the multiple biomethanes are distributed such that the biomethane(s) in the feedstock for methane reforming is/are substantially produced from wastes and/or residue, while the biomethane(s) in the fuel for methane reforming is/are generally not produced from wastes and/or residue. In certain embodiments, the multiple biomethanes are distributed such that the biomethane(s) in the feedstock for methane reforming is/are substantially produced from cellulosic material, while the biomethane(s) in the fuel for methane reforming is/are substantially produced from non-cellulosic material. In certain embodiments, the multiple biomethanes are distributed such that the biomethane(s) in the feedstock for methane reforming is/are substantially produced from fats/oils/greases, while the biomethane(s) in the fuel for methane reforming is/are generally not produced from fats/oils/greases, or vice versa. In certain embodiments, the multiple biomethanes are distributed such that the biomethane(s) in the feedstock for methane reforming has/have one or more sustainability characteristic that differ from one or more sustainability characteristic of the biomethane(s) in the fuel for methane reforming. These embodiments are particularly advantageous when the feedstock includes multiple biomethanes, the fuel includes multiple biomethanes, and/or the fuel and/or feedstock includes natural gas.

In certain embodiments, the multiple batches of biomethane are distributed such that the biomethane(s) in the feedstock for methane reforming is produced from a first feedstock type (e.g., cellulosic, waste and/or residue, fats/oils/greases, landfill, manure, food scraps, or organic fraction of municipal waste), while the biomethane(s) in the fuel for methane reforming is produced from one or more other feedstock types. Embodiments wherein the biomethane in each of the feedstock and/or fuel is substantially produced from a certain type of feedstock (e.g., at least about 90%), where the type of feedstock is associated with additional benefits under the applicable regulations, may be particularly advantageous.

In certain embodiments, the multiple batches of biomethane are distributed such that the biomethane in the feedstock for methane reforming has a first identity composition, while the biomethane(s) in the fuel for methane reforming has a second other identity composition. For purposes herein, the term “identity composition” refers to the composition in terms of the energy fractions of the constituent biomethanes produced from different feedstocks (e.g., cellulosic, waste and/or residue, fats/oils/greases, landfill, manure, food scraps, or organic fraction of municipal waste). For example, in certain embodiments, the multiple batches of biomethane are distributed such that biomethane in the feedstock for methane reforming has an identity composition corresponding to more than about 50% biomethane produced from a given type of feedstock (e.g., cellulosic, waste and/or residue, fats/oils/greases, landfill, manure, food scraps, or organic fraction of municipal waste), and biomethane in the fuel for the methane reforming has an identity composition that is 50% or less in that given type of feedstock. In certain embodiments, the multiple batches of biomethane are distributed such that biomethane in the feedstock has an identity composition that corresponds to at least about 60%, at least about 70%, at least about 80%, at least about 90%, or is about 100% of biomethane produced from a given type of feedstock.

In certain embodiments, the multiple batches of biomethane are distributed such that more megajoules of biomethane produced from cellulosic feedstock are distributed to the feedstock for methane reforming than to the fuel for methane reforming. In certain embodiments, the multiple batches of biomethane are distributed such that more megajoules of biomethane produced from wastes and/or residues are distributed to the feedstock for methane reforming than to the fuel for methane reforming. In certain embodiments, the multiple batches of biomethane are distributed such that more megajoules of biomethane produced from the organic fraction of municipal solid waste are distributed to the feedstock for methane reforming than to the fuel for methane reforming.

In certain embodiments, the multiple biomethanes are distributed such that the biomethane(s) in the feedstock for methane reforming is produced according to a first process, while the biomethane(s) in the fuel for methane reforming is produced according to another process. In certain embodiments, the multiple biomethanes are distributed such that such that the biomethane(s) in the feedstock for methane reforming is produced according to a first process that does not include CCS, while at least one biomethane(s) in the fuel for methane reforming is produced according to another process that includes CCS. In certain embodiments, at least two of the biomethanes differ from each other in terms of the anaerobic digestion process (e.g., in one process digestate is fed to an open lagoon and in the other process digestate is fed to a closed lagoon). In certain embodiments, the multiple biomethanes are distributed such that the biomethane(s) in the feedstock for methane reforming is produced according to a first process that combusts off-gas from hydrogen purification, while at least one biomethane in the fuel for methane reforming is produced according to another process that does not combust off-gas from hydrogen purification.

In certain embodiments, at least one of the batches of biomethane is distributed such that the biomethane in the feedstock has a first weighted average carbon intensity and the biomethane in the fuel has a different weighted average carbon intensity. The weighted average carbon intensity of biomethane in the feedstock can be calculated as follows:

$$\frac{{CI}_{B 1} \times M {J}_{B 1} + {CI}_{B 2} \times M {J}_{B 2} + … + {CI}_{B n} \times M {J}_{n}}{M {J}_{B 1} + M {J}_{B 2} + … + M {J}_{n}} \qquad ( 8 )$$

where CI_{B1} refers to the carbon intensity of a first biomethane, MJ_{B1} refers to an amount of the first biomethane that is feedstock (in MJ), CI_{B2} refers to the carbon intensity of a second biomethane, MJ_{B2} refers to an amount of the second biomethane that is feedstock (in MJ), CI_{Bn} refers to the carbon intensity of the nth biomethane (if more than 2 biomethanes are provided), and MJ_{Bn} refers to an amount of the nth biomethane that is feedstock (in MJ) (if more than 2 biomethanes are provided). In instances where there is only one biomethane present in the feedstock, the weighted average carbon intensity of the biomethane in the feedstock is the carbon intensity of the biomethane that is present.

When each of the biomethanes is distributed proportionally between the feedstock and fuel, the biomethane in the feedstock and fuel will generally have the same weighted average carbon intensity. In contrast, when at least one of the biomethanes is distributed disproportionally between the feedstock and fuel, the biomethane in the feedstock and fuel can have different weighted average carbon intensities. Although each feedstock may be generally associated with its own carbon intensity, the weighted average carbon intensity of biomethane in the feedstock can be compared to the weighted average carbon intensity of biomethane in the fuel for comparative purposes.

In certain embodiments, a weighted average carbon intensity of biomethane in feedstock is at least 10 gCO₂e/MJ, at least 20 gCO₂e/MJ, at least 30 gCO₂e/MJ, at least 40 gCO₂e/MJ, or at least 50 gCO₂e/MJ, lower than the weighted average carbon intensity of biomethane in fuel when calculated using GREET 2021.

In certain embodiments, the multiple biomethanes includes a first biomethane and a second biomethane, a carbon intensity of the second batch of biomethane is lower than a carbon intensity of the first batch of biomethane, and at least about 55%, at least about 60%, at least about 70%, at least about 75%, at least about 80%, at least about 85%, at least about 90%, at least about 95%, at least about 99%, or about 100% of the second batch of biomethane is distributed to the fuel. In certain embodiments, the second batch of biomethane is selected to have a carbon intensity that is not higher than about 0 gCO₂e/MJ, about −10 gCO₂e/MJ, about −20 gCO₂e/MJ, about −30 gCO₂e/MJ, about −40 gCO₂e/MJ, about −50 gCO₂e/MJ, about −60 gCO₂e/MJ, about −70 gCO₂e/MJ, about −80 gCO₂e/MJ, about −90 gCO₂e/MJ, about −100 gCO₂e/MJ, about −150 gCO₂e/MJ, or about −200 gCO₂e/MJ when calculated using GREET 2021. Alternatively, or additionally, in certain embodiments, the first batch of biomethane is selected to have a carbon intensity that is not lower than about 10 gCO₂e/MJ, about 20 gCO₂e/MJ, about 30 gCO₂e/MJ, or about 40 gCO₂e/MJ when calculated using GREET 2021.

In certain embodiments, the multiple biomethanes includes a first biomethane and a second biomethane, a quantity of the first batch of biomethane is higher than a quantity of the second batch, and at least about 55%, at least about 60%, at least about 70%, at least about 75%, at least about 80%, at least about 85%, at least about 90%, at least about 95%, at least about 99%, or about 100% of the first batch of biomethane is distributed to the feedstock.

In certain embodiments, the multiple biomethanes are distributed to increase a percentage of the hydrogen produced that is below a certain carbon intensity (e.g., below a target intensity) relative to when each of the multiple biomethanes are distributed proportionally. In certain embodiments, the multiple biomethanes are distributed to increase a percentage of the hydrogen produced that qualifies for at least one GHG emissions based incentive relative to a proportional distribution of the at least two batches of biomethane. For example, in certain embodiments, the distribution is selected such that the percentage of hydrogen produced that qualifies as clean hydrogen and/or for at least one GHG emissions based incentive is at least about 45%, at least about 50%, at least about 60%, at least about 70%, at least about 80%, at least about 90%, at least about 95%, or about 100%. Clean hydrogen may be defined, for example, as hydrogen produced with a carbon intensity equal to or less than some predetermined carbon intensity (e.g., set by regulators) and/or that meets or exceeds some predetermined carbon emission reductions (e.g., hydrogen made with a process that emits at least about 50% less carbon dioxide than the use of steam methane reforming from natural gas). As illustrated in the examples, how and where the biomethanes are used in the hydrogen production process can determine the carbon intensity of the hydrogen produced and/or whether it qualifies as clean hydrogen and/or for any GHG emissions based incentives. Providing feedstock and fuel having biomethane contents with different weighted average carbon intensities is particularly advantageous over approaches that simply treat carbon emission reductions from multiple biomethanes as cumulative and/or independent of where they are used.

In certain embodiments, the distribution is selected such that at least a portion of the quantity of hydrogen produced achieves a GHG reduction of at least about 45%, at least about 50%, at least about 60%, at least about 70%, at least about 80%, at least about 90%, or at least about 95%, relative to if the feedstock and fuel were entirely non-renewable. In certain embodiments, the distribution is selected such that at least a portion of the quantity of hydrogen produced achieves a GHG reduction of at least about 45%, at least about 50%, at least about 60%, at least about 70%, at least about 80%, at least about 90%, or at least about 95%, relative to a baseline set by the applicable regulatory agency based on conventional steam methane reforming of non-renewable natural gas.

### Reducing the Carbon Intensity

In certain embodiments, the carbon intensity and/or lifecycle GHG emissions of the hydrogen and/or product produced therefrom is reduced by including carbon capture and storage (CCS). Carbon capture and storage (CCS) is a climate change mitigation technology that leads to a reduction in atmospheric carbon dioxide relative to the option of not using the technology. In general, CCS refers to one or more processes wherein carbon dioxide is captured from the atmosphere, or captured from a process that otherwise would release it to the atmosphere, and wherein the captured carbon is stored and/or used in a way that reduces the level of carbon dioxide in the atmosphere. In general, the CCS may be part of biomethane production and/or hydrogen production. With regard to the former, the carbon emission reductions from CCS may be reflected in the carbon intensity of the biomethane(s).

In certain embodiments, the CCS includes capturing and/or storing carbon from at least one of the biomethane production processes. For example, carbon dioxide produced during biomethane production can be captured (e.g., as part of biogas upgrading and/or separate from biogas upgrading) and provided for storage (e.g., injected into a carbon dioxide pipeline). Alternatively, or additionally, carbon-containing material obtained or derived from residue from biomethane production (e.g., digestate, char, etc.) can be processed and provided for storage. Processing such residue can, for example, produce gas containing carbon dioxide, liquid such as bio-oil, or solid such as biochar, which can be sequestered (e.g., provided for substantially permanent storage and/or use in beneficial applications (e.g., that consume carbon dioxide to make a product).

In certain embodiments, the CCS includes capturing and/or storing carbon dioxide produced from hydrogen production (e.g., from the syngas produced by subjecting feedstock to methane reforming or from the flue gas produced by combusting fuel for providing heat for the methane reforming).

In general, the carbon dioxide can be captured using any suitable separation technology, or if the carbon dioxide is relatively pure, capturing the carbon dioxide can simply refer to collecting the carbon dioxide (e.g., in a pipe). It can be particularly advantageous to use gas separation techniques that provide a relatively pure carbon dioxide stream. Such techniques may for example, include vacuum PSA (VPSA), absorption processes (e.g., based on amines), and/or cryogenic separations (e.g., using temperatures below −10° C. or below −50° C.). In general, the carbon dioxide can be captured using systems provided for hydrogen purification and/or separate from hydrogen purification.

In general, the carbon dioxide can be provided for storage according to any suitable technology or combination of technologies that prevents and/or delays the release of the captured carbon dioxide, or an equal quantity of carbon dioxide displaced physically by the captured carbon dioxide, to the atmosphere. For example, storage of the captured carbon dioxide can include injecting it into a carbon dioxide pipeline configured to transport the carbon dioxide to a location where it can be sequestered in a subsurface formation (e.g., trapped it in a geological formation, such as a saline aquifer, oil and natural gas reservoir, unmineable coal seam, organic-rich shale, or basalt formation). Storage of the captured carbon dioxide can also be part of carbon capture, utilization, and storage, or CCUS. CCUS technologies encompass the use of the captured carbon dioxide. For example, CCUS can include using the captured carbon dioxide, or an equal quantity of carbon dioxide displaced physically by the captured carbon dioxide, for enhanced oil recovery (EOR). CCUS technologies may include the use of the captured carbon dioxide, or an equal quantity of carbon dioxide displaced physically by the captured carbon dioxide, for producing a product (e.g., the carbon dioxide can be stored within the product). Such products may include building materials such as cement, concrete, or aggregates, chemicals, fuels, and/or food and beverages. As will be understood by those skilled in the art, it can be advantageous for the CCS technology to be selected such that it is recognized by the applicable regulatory authority for reducing lifecycle GHG emissions and/or mitigating climate change. For example, some regulations may require storage to have a maximum leakage rate (e.g., monitoring of carbon dioxide leakage from storage for a certain time period may be mandatory).

The term “CCS”, as used herein, can refer to CCS and/or CCUS. As will be understood by those skilled in the art, the selection of the storage may be dependent on the applicable regulations (e.g., used to calculate lifecycle GHG emissions and/or qualify the hydrogen, or fuel, fuel intermediates, or products produced from the hydrogen, for credits).

### Credits

In certain embodiments, the method(s) of the present disclosure includes generating, obtaining, or providing credits (e.g., associated with the hydrogen and/or fuel, fuel intermediates, or products produced from the hydrogen). Credits can be used to incentivize renewable products and/or products associated with reduced carbon or GHG emissions (e.g., fuels used in the transportation sector). For example, credits such as fuel credits can be used to demonstrate compliance with some government initiative, standard, and/or program, where the goal is to reduce GHG emissions (e.g., reduce carbon intensity in transportation fuels as compared to some baseline level related to conventional petroleum fuels) and/or produce a certain amount of biofuel (e.g., produce a mandated volume or a certain percentage of biofuels). The target GHG reductions and/or target biofuel amounts may be set per year or for a given target date. Some non-limiting examples of such initiatives, standards, and/or programs include the Renewable Fuel Standard Program (RFS2) in the United States, the Renewable Energy Directive (RED II) in Europe, the Fuel Quality Directive in Europe, the Renewable Transport Fuel Obligation (RTFO) in the United Kingdom, and/or the Low Carbon Fuel Standards (LCFS) in California, Oregon, or British Columbia). Credits can also be used to incentivize other products associated with reduced carbon or greenhouse gas emissions, such as for example, producer or production credits for clean hydrogen or credits for products made using clean hydrogen.

The term “credit”, as used herein, refers to any rights or benefits relating to carbon or GHG emission reductions, including but not limited to rights to credits, revenues, offsets, GHG gas rights, tax benefits, government payments, or similar rights or benefits related to or arising from emission reductions, trading, or any quantifiable benefits (including recognition, award or allocation of credits, allowances, permits or other tangible rights), whether created from or through a government authority, a private contract, or otherwise. A credit can be a certificate, record, serial number or guarantee, in any form, including electronic, which evidences production of a quantity of a product (e.g., hydrogen or fuel, fuel intermediate, or product produced from the hydrogen) meeting certain life cycle GHG emission reductions relative to a baseline (e.g., a gasoline baseline) set by a government authority. Credits for low carbon intensity hydrogen may be set by regulatory authority and provided in many forms, e.g., producer credits and the like. Non-limiting examples of fuel credits include RINs and LCFS credits. A Renewable Identification Number (or RIN), which is a certificate that acts as a tradable currency for managing compliance under the RFS2, may be generated for each gallon of biofuel (e.g., ethanol, biodiesel, etc.) produced. A Low Carbon Fuel Standard (LCFS) credit, which is a certificate which acts as a tradable currency for managing compliance under California's LCFS, may be generated for each metric ton (MT) of CO₂ reduced. Credits for clean or low CI hydrogen may be set by the appropriate regulatory authority and provided in many forms, e.g., producer or production credits and the like. In certain embodiments, the method(s) includes generating, obtaining, or providing producer or production credits for clean hydrogen or credits for products made using clean hydrogen.

In general, the requirements for obtaining, generating, or causing the generation of credits can vary by country, the agency, and or the prevailing regulations in/under which the credit is generated. In some cases, credit generation may be dependent upon a compliance pathway (e.g., predetermined or applied for) and/or the product (e.g., hydrogen and/or fuel, fuel intermediates, or products produced from the hydrogen) meeting a predetermined GHG emission threshold. For example, with regard to the former, the RFS2 categorizes biofuel as cellulosic biofuel, advanced biofuel, renewable biofuel, and biomass-based diesel. With regard to the latter, to be a renewable biofuel under the RFS2, corn ethanol should have lifecycle GHG emissions at least 20% lower than an energy-equivalent quantity of gasoline (e.g., 20% lower than the 2005 EPA average gasoline baseline of 93.08 gCO₂eq/MJ). In low carbon-related fuel standards, biofuels may be credited according to the carbon reductions of their pathway. For example, under California's LCFS, each biofuel is given a carbon intensity score indicating their GHG emissions as grams of CO₂ equivalent per megajoule (MJ) of fuel, and credits are generated based on a comparison of their emissions reductions to a target or standard that may decrease each year (e.g., in 2019, ethanol was compared to the gasoline average carbon intensity of 93.23 gCO₂eq/MJ), where lower carbon intensities generate proportionally more credits.

In certain embodiments of the disclosure, the method includes monitoring inputs and/or outputs from each of the biomethane production, hydrogen production, and/or CCS. In this case, each of the inputs is a material input or energy input and each of the outputs is a material output or an energy output. Monitoring inputs and/or outputs of these processes may facilitate calculating and/or verifying GHG emissions of the process, calculating and/or verifying carbon intensity of the hydrogen or a fuel, fuel intermediate, or product produced using the hydrogen, may facilitate credit generation (e.g., based on volumes of fuel produced), and/or may facilitate determining renewable content (e.g., when co-processing renewable and non-renewable fuels). Monitoring can be conducted over any time period (e.g., monthly statements, etc.). Monitoring can be conducted in conjunction with and/or using any suitable technology or combination of technologies that enables measurement of material and/or energy flows.

### Use of Hydrogen Produced

The method(s) of the present disclosure generally relate to producing hydrogen, or to a method of producing a fuel, fuel intermediate, or product that includes hydrogen production (e.g., production of a product using hydrogen). In certain embodiments, the method(s) include using the hydrogen as a fuel, or producing a product (e.g., fuel, fuel intermediate, and/or chemical product) using the hydrogen produced. For example, the method(s) can include methods of producing hydrogen (e.g., renewable hydrogen), methods of producing fuel (e.g., one or more fuels such as hydrogen, gasoline, diesel, jet fuel, methanol, ethanol), chemical product (e.g., methanol, ammonia, fertilizer, etc.), or intermediates (e.g., methanol, hydrogen, ammonia, ethanol, etc.).

In certain embodiments, the hydrogen is used in the hydroprocessing (e.g., hydrocracking and/or hydrotreating) of crude-oil derived liquid hydrocarbon such that the hydrogen (i.e., at least the renewable hydrogen) is incorporated into a crude-oil derived liquid hydrocarbon to produce, for example, gasoline, diesel, and/or jet fuel having renewable content (e.g., see U.S. Pat. Nos. 8,658,026, 8,753,854, 8,945,373, 9,040,271, 10,093,540, 10,421,663 10,723,621 and 10,981,784, which are incorporated herein by reference). The term “crude oil derived liquid hydrocarbon”, as used herein, refers to any carbon-containing material obtained and/or derived from crude oil that is liquid at standard ambient temperature and pressure. The term “crude oil”, as used herein, refers to petroleum extracted from geological formations (e.g., in its unrefined form). Crude oil includes liquid, gaseous, and/or solid carbon-containing material from geological formations, including oil reservoirs, such as hydrocarbons found within rock formations, oil sands, or oil shale. The term “renewable content”, as used herein, refers to the portion of the fuel(s) that is recognized and/or is treated as renewable (e.g., a biofuel) under applicable regulations. As will be understood by those skilled in the art, the quantification of the renewable content can be determined using any suitable method and can be dependent upon the applicable regulations.

In certain embodiments, the hydrogen is used to produce ammonia in a Haber-Bosch process. In the Haber-Bosch process, which is well-known to those skilled in the art, nitrogen is converted to ammonia according to the following reaction:

N₂+3H₂→2NH₃ (9)

The reaction is conducted under high temperatures and pressures with a metal catalyst. Ammonia has an important role in the agricultural industry for production of fertilizers. Ammonia may also be used as an energy carrier for energy storage and transportation.

In certain embodiments, the hydrogen is provided as a product (e.g., for use in a fuel cell or a fuel). For example, the hydrogen can be used for transportation purposes, for generating electricity, and/or for use in district heating.

In certain embodiments, the hydrogen is provided as feedstock in a production process that produces a fuel, fuel intermediate, chemical product, or any combination thereof. A fuel refers to a material (e.g., solid, liquid, or gaseous), which may contain carbon, that can be combusted to produce power and/or heat (e.g., may be a transportation or heating fuel). A fuel intermediate is a precursor used to produce a fuel by a further conversion process, such as by a biologic conversion, a chemical conversion, or a combination thereof. A chemical product refers to a chemical compound used in a production process or a product such as a commodity. An example of a chemical product produced from hydrogen is fertilizer.

In certain embodiments, the hydrogen is provided as feedstock to produce a fuel selected from long-haul trucking fuel (e.g., diesel), shipping fuel (e.g., heavy fuel oil), aviation fuel (e.g., kerosene, jet fuel) or district heating fuel.

In certain embodiments, the hydrogen is used to produce one or more alcohols via gas fermentation using known processes. In gas fermentation, which is well-known to those skilled in the art, a gas mixture typically containing hydrogen with carbon dioxide and/or carbon monoxide is fed into a fermentation tank. In this embodiment, the carbon monoxide in the syngas functions as a substrate for the biologic conversion, which utilizes microorganisms or other biocatalysts. For example, acetogenic microorganisms can be used to produce a fermentation product from carbon monoxide. The production of ethanol by the acetogenic microorganisms proceeds through a series of biochemical reactions.

In certain embodiments, the hydrogen is used to produce methanol. For example, methanol can be produced by directly hydrogenating carbon dioxide with hydrogen using Cu/ZnO-based catalysts. Alternatively, hydrogen can be used to produce methanol according to the following reactions:

CO₂+H₂→CO+H₂O (reverse water gas shift) (10)

CO+2H₂→CH₃OH (11)

The methanol can be used as a fuel (e.g., mixed with gasoline) or can be used to produce a fuel (e.g., biodiesel).

In certain embodiments, the hydrogen is used to produce gasoline, diesel, and/or waxes using the Fischer-Tropsch process. The Fischer-Tropsch process refers to a collection of chemical reactions that converts syngas into liquid hydrocarbons, typically in the presence of metal catalysts under elevated pressures and temperatures. The Fischer-Tropsch process is well known. In the embodiments including a Fischer-Tropsch process, the hydrogen may be used to supplement another gas feed containing carbon monoxide and/or carbon dioxide in order to provide the required H₂ to CO ratio (e.g., about 2).

While producing a hydrogen product, such as a renewable hydrogen product, is advantageous, it is particularly advantageous when the renewable hydrogen is used as feedstock for a production process (e.g., to produce a fuel, fuel intermediate, or chemical product). It can be particularly advantageous when the renewable hydrogen is used as feedstock for producing a transportation fuel. Using the renewable hydrogen in a production process can reduce GHG emissions associated with production process, and when the production process produces a fuel, can impart renewable content to the fuel and/or reduce the carbon intensity of the fuel. The GHG reductions can be significant, particularly when the renewable hydrogen has a negative carbon intensity.

### EXAMPLE

A hydrogen production process produces 100 MJ hydrogen from every 130 MJ of feed. Of the 130 MJ of feed, 100 MJ is feedstock and 30 MJ is fuel. Two batches of biomethane are provided for the hydrogen production. The first batch is 100 MJ of a first biomethane having a carbon intensity of 8 gCO₂eq/MJ. The second batch is 30 MJ of a second biomethane having a carbon intensity of −50 gCO₂eq/MJ.

Without selectively distributing each of the first and second biomethanes between feedstock and fuel, about 77% of the biomethane in the feedstock will be the first biomethane, while the remaining 23% is the second biomethane. The weighted average carbon intensity of biomethane in each of the feedstock and fuel is −5.3 gCO₂eq/MJ (e.g., 0.77*8 gCO₂eq/MJ+0.23*(−50 gCO₂eq/MJ)). Since two different feedstocks are used (e.g., the first and second biomethanes), two different hydrogens are produced.

The 77 MJ batch of hydrogen produced from the first biomethane can have GHG emissions associated with the feedstock (e.g., 616 gCO₂eq=77 MJ*8 gCO₂eq/MJ) and a GHG emissions credit associated with the fuel (e.g., −122 gCO₂eq=0.77*30 MJ*(−5.3 gCO₂eq/MJ)), for a total of about 494 gCO₂eq (i.e., which corresponds to about 4.9 gCO₂eq/MJ of hydrogen).

The 23 MJ batch of hydrogen produced from the second biomethane can have a GHG emissions credit associated with the feedstock (e.g., −1150 gCO₂eq=23 MJ*(−50 gCO₂eq/MJ)) and a GHG emissions credit associated with the fuel (e.g., −36.6 gCO₂eq=0.23*30 MJ*(−5.3 gCO₂eq/MJ)), for a total of about −1186.6 gCO₂eq (i.e., which corresponds to about −11.9 gCO₂eq/MJ of hydrogen).

Assuming that the carbon emissions from the biomethanes must be less than 4 gCO₂eq/MJ of hydrogen in order to meet or exceed a certain lifecycle GHG reduction, then absent any CCS from hydrogen production, only about 23% of the hydrogen produced from the process will meet or exceed the certain lifecycle GHG reduction.

In contrast, consider the disproportional distribution where 100 MJ of the first biomethane is designated as feedstock and 30 MJ of the second biomethane is designated as fuel. In this case, the weighted average carbon intensity of the feedstock is 8 gCO₂eq/MJ and the weighted average carbon intensity of biomethane in the fuel is −50 gCO₂eq/MJ. About 100% of the biomethane in the feedstock will be the first biomethane, thereby providing a single batch of hydrogen. This 100 MJ batch of hydrogen can have carbon emissions associated with the feedstock (e.g., 800 gCO₂eq=100 MJ*8 gCO₂eq/MJ) and a carbon emissions credit associated with the fuel (e.g., −1500 gCO₂eq=30 MJ*(−50 gCO₂eq/MJ)), for a total of about −700 gCO₂eq (i.e., which corresponds to about −7 gCO₂eq/MJ of hydrogen). Advantageously, this disproportional distribution can result in about 100% of the hydrogen produced from the process meeting or exceeding the certain lifecycle GHG reduction (e.g., corresponding to 4 gCO₂eq/MJ).

In the above example, it is assumed that the hydrogen is produced from a stand-alone hydrogen plant based on steam methane reforming that operates without CCS. The example also assumed that there are no significant carbon emissions from other sources (e.g., methane loss in pipeline, transportation emissions, etc.). It will be understood by those skilled in the art that a disproportional distribution of two biomethanes can also be used in hydrogen production that is within an oil refinery or other production facility (e.g., ammonia), which may, for example, produce a fuel/product having reduced carbon emissions.

If course, the above embodiments have been provided as examples only. It will be appreciated by those of ordinary skill in the art that various modifications, alternate configurations, and/or equivalents will be employed without departing from the scope of the invention. Accordingly, the scope of the invention is therefore intended to be limited solely by the scope of the appended claims.

1. A method of producing hydrogen, the method comprising:
(a) providing at least two batches of biomethane for producing hydrogen from a hydrogen production process, the at least two batches of biomethanes including a first batch of a first biomethane and a second batch of a second biomethane, the first and second biomethanes being produced from different feedstocks, being produced from different processes, having different carbon intensities, or any combination thereof, the hydrogen production process comprising:
(i) subjecting feedstock to methane reforming to produce syngas,
(ii) combusting fuel for producing heat for the methane reforming, and
(iii) subjecting the syngas or a gas derived from the syngas to a hydrogen purification process; and
(b) generating a quantity of hydrogen from the hydrogen production process using the at least two batches of biomethane, at least a portion of the first batch distributed to the feedstock, at least a portion of the second batch distributed to the fuel, a distribution of the at least two batches of biomethane between the feedstock and fuel providing the feedstock with biomethane having a first fractional make-up and the fuel with biomethane having a second fractional make-up, the first fractional make-up being different from the second fractional make-up.

2. The method according to claim 1, wherein the distribution of the at least two batches of biomethane increases a percentage of the quantity of hydrogen that qualifies for at least one greenhouse gas emissions based incentive relative to a proportional distribution of the at least two batches of biomethane.

3. The method according to claim 1, wherein the distribution of the at least two batches of biomethane between the feedstock and fuel provides the feedstock with biomethane having a first weighted average carbon intensity and the fuel with biomethane having a second weighted average carbon intensity, the first weighted average carbon intensity being different from the second weighted average carbon intensity.

4. The method according to claim 3, wherein the first weighted average carbon intensity is higher than the second weighted average carbon intensity.

5. The method according claim 1, wherein a carbon intensity of the second batch of biomethane is lower than a carbon intensity of the first batch of biomethane, and wherein at least 60% of the second batch of biomethane is distributed to the fuel.

6. The method according to claim 1, wherein a carbon intensity of the second batch of biomethane is lower than a carbon intensity of the first batch of biomethane, and wherein at least 90% of the second batch is distributed to the fuel.

7. The method according to claim 1, wherein the distribution of the at least two batches of biomethane between the feedstock and fuel provides the feedstock with biomethane having a first identity composition and the fuel with biomethane having a second identity composition, the first identity composition being different from the second identity composition.

8. The method according to claim 1, wherein the distribution of the at least two batches of biomethane between the feedstock and fuel provides more megajoules of biomethane produced from cellulosic feedstock to the feedstock for methane reforming than to the fuel for methane reforming.

9. The method according to claim 1, wherein the first batch of biomethane is larger on an energy basis than any of the other batches in the at least two batches of biomethane, and wherein at least 60% of the first batch on an energy basis is distributed to the feedstock.

10. The method according to claim 1, wherein the first batch of biomethane is larger on an energy basis than any of the other batches in the at least two batches of biomethane, and wherein at least 90% of the first batch on an energy basis is distributed to the feedstock.

11. The method according to claim 1, wherein the feedstock, the fuel, or a combination thereof, comprises non-renewable natural gas, refinery gas, or a combination thereof.

12. The method according to claim 1, wherein the feedstock consists essentially of the first batch of biomethane.

13. The method according to claim 1, wherein the fuel comprises the second batch of biomethane and a third batch of biomethane.

14. The method according to claim 1, wherein the first batch of biomethane is withdrawn from a natural gas distribution system, and wherein the second batch of biomethane is withdrawn from a vessel transported by vehicle.

15. The method according to claim 1, wherein the methane reforming includes steam methane reforming, and wherein (ii) comprises combusting fuel for producing heat for the steam methane reforming.

16. The method according to claim 1, wherein the hydrogen production process includes capturing carbon dioxide as part of a carbon capture and storage process.

17. The method according to claim 1, wherein at least a portion of the quantity of hydrogen achieves a greenhouse gas reduction of at least 60% relative to a baseline hydrogen production process where the feedstock and fuel are natural gas.

18. The method according to claim 1, wherein at least a portion of the quantity of hydrogen achieves a greenhouse gas reduction of at least 95% relative to a baseline hydrogen production process where the feedstock and fuel are natural gas.

19. The method according to claim 1, wherein the hydrogen production process comprises recycling an off-gas from the hydrogen purification process for use as fuel in (ii).

20. The method according to claim 1, wherein the each of the at least two batches of biomethane is withdrawn from a natural gas distribution system.

21. A method of producing hydrogen, the method comprising:
(a) providing at least two batches of biomethane for producing hydrogen from a hydrogen production process, the at least two batches of biomethanes including a first batch of a first biomethane having a first carbon intensity and a second batch of a second biomethane having a second carbon intensity, the first and second biomethanes being different, the hydrogen production process comprising:
(i) subjecting feedstock to steam methane reforming to produce syngas,
(ii) combusting fuel for producing heat for the steam methane reforming, and
(iii) subjecting the syngas or a gas derived from the syngas to a hydrogen purification process; and
(b) generating a quantity of hydrogen from the hydrogen production process using the at least two batches of biomethane, at least a portion of the first batch distributed to the feedstock, at least a portion of the second batch distributed to the fuel, a distribution of the at least two batches of biomethane providing the feedstock with biomethane having first weighted average carbon intensity and the fuel with biomethane having a second weighted average carbon intensity, the first weighted average carbon intensity being different than the second weighted average carbon intensity.

22. The method according to claim 21, wherein the first weighted average carbon intensity is higher than the second weighted average carbon intensity.

23. (canceled)

24. A method of producing fuel, fuel intermediate, product, or any combination thereof, the method comprising:
(a) providing at least two batches of biomethane, the at least two batches of biomethane including a first batch of a first biomethane and a second batch of a second biomethane, the first and second biomethanes being produced from different feedstocks, being produced from different processes, having different carbon intensities, or any combination thereof;
(b) generating syngas using the at least two batches of biomethane, the generating comprising methane reforming; and
(c) distributing at least a portion of the first batch as feedstock for the methane reforming and at least a portion of the second batch as fuel for producing heat for the methane reforming,
said distributing providing the feedstock with biomethane having a first fractional make-up and the fuel with biomethane having a second fractional make-up, the first fractional make-up being different from the second fractional make-up.
